(19) United States
(12) Patent Application Publication
(10) Pub. No.: US 2013/0131655 A1
(43) Pub. Date: May 23, 2013

(54) BREAST RECONSTRUCTION OR AUGMENTATION USING COMPUTER-MODELED DEPOSITION OF PROCESSED ADIPOSE TISSUE

(71) Applicant: Allergan, Inc., Irvine, CA (US)

(72) Inventors: Gino Rigotti, Verona (IT); Guido Baroni, Monza (IT)

(73) Assignee: ALLERGAN, INC., Irvine, CA (US)

(21) Appl. No.: 13/736,811

(22) Filed: Jan. 8, 2013

Related U.S. Application Data
(62) Division of application No. 12/334,140, filed on Dec. 12, 2008, now abandoned.
(60) Provisional application No. 61/013,864, filed on Dec. 14, 2007.

(51) Int. Cl. A61F 2/12

(52) U.S. Cl. CPC A61F 2/12
USPC 606/8

(57) ABSTRACT

A tissue transfer method for reconstruction and augmentation of soft tissue. The method includes harvesting adipose tissue from a patient. The harvested tissue is processed via centrifugation to isolate a purified subset of the adipose tissue including separating and removing a substantial amount of triglycerides from the harvested adipose tissue. The centrifugation may be performed to cause separation of water from the purified adipose tissue and to cause separation of oil from mature adipocytes. Specifically, the spin rates may be selected to be high enough to cause lesions in the mature adipocytes that results in the release of the oil. The method continues with implanting the purified adipose tissue into the patient at a breast or other area identified for reconstruction or augmentation. The implanting is performed based on an injection pathway model that defines injection point locations and a number of injection pathway directions from each point.

### RELATED APPLICATION

This application is a divisional of U.S. patent application Ser. No. 12/334,140, filed Dec. 12, 2008, which claims priority to U.S. Provisional Patent Application No. 61/013,864, filed Dec. 14, 2007 and each of which is incorporated herein in its entirety by this specific reference.

### BACKGROUND OF THE INVENTION

1. Field of the Invention

The present invention relates, in general, to tissue reconstruction and augmentation such as reconstructing or augmenting breast tissue, and, more particularly, to methods and systems for reconstructing breasts or augmenting breasts using adipose tissue such as with selective depositions or injections of processed or purified adipose tissue (e.g., adipose-derived adult stem cells (ADAS) or adipose tissue with a high concentration of ADAS) using computerized models or computer assisted surgical planning.

2. Relevant Background

Each year, hundreds of thousands of women undergo breast surgery to augment or to reconstruct breast tissue. The surgery may be cosmetic such as to increase the size of the breast through augmentation. In many other cases, the surgery follows a therapeutic surgery or therapy that has resulted in the removal or damage of breast tissue. This type of breast surgery may be considered reconstructive surgery that attempts to provide the patient with a breast that has the shape and texture of their breast before therapy. For example, a woman diagnosed with breast cancer and treatment may involve a radical mastectomy to remove the breast or a lumpectomy to remove a smaller portion of the breast. Breasts or mammary glands largely composed of adipose cells or tissue (e.g., body tissue that stores fat). Instead of or in addition to tissue removal, treatments for breast cancer may involve radiotherapy that typically causes permanent damage to the breast tissue including the adipose tissue such as by inducing fibrosis or lesions. The damage may even worsen over time resulting in the patient developing ulcers or other problems. Presently, the two main augmentation and reconstruction techniques are to install a breast implant or to transfer tissue into the breast, but each of these techniques has limitations that have led to continued research by the medical community to find better solutions to this ongoing problem.

Breast implants continue to be the most popular reconstruction and augmentation technique because they can typically be installed relatively quickly and effectively by most plastic surgeons. A breast implant is a prosthesis that is used to enlarge the size of a woman's breasts. For example, an elastomer or silicone shell of a desired size and shape may be filled with sterile saline or filled with silicone gel, and the reconstruction or augmentation procedure involves making an incision in the patient and inserting the filled shell. While the surgical procedure is relatively straightforward, many patients have experienced serious complications. Implants are generally not lifetime devices, and most patients will require additional surgeries such as after the implant ruptures causing the implant to leak and deflate. As an immune response, the patient's body may form capsules of tightly-woven collagen fibers around the implant (e.g., capsular contracture) which can result in the appearance and texture of the implant being altered and cause the patient pain. The patient may also develop autoimmune issues or infections. Additionally, symmetry may be lost after the surgery if the implant moves or becomes displaced.

Many in the medical industry prefer to use tissue transfer when performing reconstruction and augmentation of breasts and other portions of the body such as the face, buttocks, and other areas of soft tissue. For example, surgeons often prefer to refill a breast void or envelope with a patient's own adipose tissue. However, trials involving large mass transfer of tissue such as adipose tissue have not been particularly successful and technical challenges have made many physicians or surgeons wary of these techniques to the point that they more often recommend the use of implants. Adipose tissue is found in many places in the human body and is found in excess amounts that allow it to be harvested from most patients without creating contour deformities or other problems. The transplanting or transfer of adipose tissue typically begins by liposuctioning the abdominal region or a patient's thighs. The harvested or aspirated adipose tissue is then inserted into the breast area of the patient using small gauge needles which may be thought of as lipoinjection.

Unfortunately, autologous fat transplantation or the direct transfer of adipose tissue has so far yielded poor results with some estimating that a reduction in the volume of the transferred tissue is up to fifty percent or more. The reduction in tissue volume may be the result of insufficient re-vascularization with some research indicating that necrosis occurs due to the lack of blood supply which may also result in cysts, fibrosis, or calcification. In some cases, the body tends to absorb at least a portion of the transplanted fat or adipose tissue in a few months. Another problem with transferring adipose tissues is that mature adipocytes found in the adipose tissue are easily damaged during aspiration by the mechanical forces used in liposuction and other harvesting methods. Mature adipocytes are the cells that primarily compose adipose tissue and include one or more lipids surrounded by a ring or sheet of cytoplasm, and mesenchymal stem cells can differentiate into adipocytes and are found in the adipose tissue that is harvested. However, the damaged adipocytes do not continue to thrive and grow when transplanted, and the small amount of stem cells is not effective in treating damaged tissues such as tissue damaged by radiotherapy.

Progress is being made in addressing tissue transfer problems, but existing processes are typically relatively complicated to implement, are expensive, and/or have not proven effective. For example, research is proceeding in using preadipocytes (i.e., precursor cells that differentiate into mature adipocytes) and using adult stem cells that both grow relatively quickly using standard cell culture technologies. To reconstruct or augment adipose tissue in a breast, the preadipocytes or adult stem cells are typically grown in a biodegradable matrix or the like to try to ensure that the new or grown adipose tissue is well vascularized. Additionally, tissue engineers have used support structures or scaffolds formed of biodegradable materials to provide a final tissue shape and a physical support for the anchorage-dependent cells to migrate and proliferate. The scaffolds may be either implanted such as a porous biodegradable polymer foam or injected such as a hydrogel. Growth factors may also have to be added to the matrix to provide a microenvironment that encourages tissue formation. Other augmentation procedures involve harvesting a patient's own adipose tissue, reserving a first portion of the harvested tissue for later transplanting, processing a second portion of the harvested tissue to extract stem cells, mixing the extracted stem cells into the portion of the adipose tissue to be transplanted to try to increase the stem cell concentration of this tissue (i.e., try to replace damaged adipocytes), and lipoinjecting the adipose tissue having the additional stem cells. While providing significant progress, these tissue transfer techniques are very expensive, are technically challenging and only feasible in elite facilities and by highly trained and skilled physicians, and may require long tissue growth periods and recovery periods.

Another ongoing challenge with tissue transfer as compared to breast implants is how best to position or distribute the new tissue. Some existing surgical methods simply call for transplanting or injecting adipose or other tissue into the breast in one or more relatively large clumps. This often results in poor blood supply which can result in necrosis of the tissue. Such clump injection also results in noticeable lumps in the reconstructed or augmented breast. Some surgeons follow a procedure of making several injections of the implanted tissue in layers from the base of the breast upward toward the nipple. While providing a somewhat better distribution of the implanted tissue, it has not provided uniform distribution (e.g., may result in a number of smaller lumps at the injection points) with its effectiveness varying widely with the surgeon and from patient to patient. In some reconstruction procedures, a tissue expander is first implanted into the patient and a biodegradable matrix seeded with cells that are intended to form new tissue is injected at a single point. The tissue grows to fill a void around the expander as it is gradually or periodically deflated or reduced in volume. This process provides some improvements in tissue distribution but requires the use of a tissue expander and requires repeated clinical visits to adjust the tissue expander.

There remains a need for improved methods and tools for performing soft tissue augmentation and reconstruction. Preferably, such methods and tools would be well-suited for use in breast surgery such as reconstructing a breast after radiotherapy treatment.

### SUMMARY OF THE INVENTION

The present invention addresses the above problems by providing a method for performing tissue transfer including an improved technique for preparing or purifying the tissue prior to implant and an improved technique for achieving a more uniform distribution of the tissue to control lumping and provide enhanced vascularization. Briefly, embodiments of the invention include harvesting or removing adipose tissue from a patient or donor and then purifying the harvested tissue prior to implant at a site or area of the same or a different patient that has been identified for augmentation or, more typically, for reconstruction (such as after radiotherapy has damaged soft tissue). The purification generally involves centrifugation at spin speeds and times selected to not only separate water and triglycerides from the harvested adipose tissue but to also damage or cause lesions in a significant portion of the mature adipocytes in adipose tissue. The water, triglycerides, and oil from the damaged, mature adipocytes (as well as other byproducts or tissue components) are separated from the now “purified” adipose tissue, which is injected or lipoinjected into the tissue injection site or area on the patient. Uniform distribution is achieved by performing the injecting or implant of tissue based on an injection pathway model that defines the location of a plurality of injection points and directions of one or more injection pathways used at each injection point. The injection pathway model is generated by first creating a surface model of the injection area or site on the patient (e.g., a 3D model of a breast to be reconstructed or augmented) and then optimizing distribution based on input optimization variables or parameters such as number of injection points, number of injection pathways at each point, and length of injection pathways. The actual implant is often monitored to provide real time guidance to the surgeon and to determine actual injection points and pathways to allow computation of achieved versus modeled tissue distribution in the patient.

The present tissue transfer method recognizes that harvesting of adipose tissue results in significant damage to mature adipocytes and that the main active component is adipose-derived adult stem cells (ADAS). Others have attempted to try to increase the concentration of surviving mature adipocytes whereas the present method uses a purification method that actually further damages the mature adipocytes hastening their removal after implant and also removes triglycerides, with initial results of the use of the purified adipose tissue showing a significant increase in the quality of the results including healing of irradiated areas in treated patients.

More particularly, a tissue transfer method is provided that is useful for reconstruction and augmentation of soft tissue. The method includes harvesting or removing a volume of adipose tissue from a patient. The harvested tissue is processed via centrifugation or other separation techniques to isolate a purified subset of the adipose tissue. This processing includes separating and removing a substantial amount of triglycerides from the harvested adipose tissue. The method continues with implanting the purified adipose tissue into the patient at an area or site (such as the patients face or a breast) identified for reconstruction or augmentation. The centrifugation may be performed at one or more spin rates and spin times to cause separation of water from the purified adipose tissue and also to cause separation of oil from mature adipocytes. Specifically, the spin rates may be selected to be high enough to cause lesions or other damage in the mature adipocytes that results in the release of the oil. For example, the spin rates may be in the range of about 1000 to about 4000 RPM and typically is between about 1500 and about 2700 RPM. The implanting in some embodiments is performed based on an injection pathway model, and in these embodiments, the tissue transfer method includes generating the injection pathway model including: preparing a surface model of the injection area or site; selecting or inputting optimization variables such as number injection points to use, injection path lengths, and number injection pathways at each injection point; performing an optimization algorithm for uniform distribution of tissue in the surface model based on the optimization variables; and providing the injection pathway model that includes a location of each of the injection points in the surface model and a direction of each of the injection pathways from each of the injection points. The method may yet further include monitoring the implanting based on the injection pathway model to determine injection pathways followed during the implanting to identify variance from the model and to determine actually achieved tissue distribution for the patient.

### BRIEF DESCRIPTION OF THE DRAWINGS

FIGS. 1A-1C illustrate methods of the invention for harvesting adipose tissue from a patient, processing or purifying the harvested tissue; and transferring the purified tissue to a breast of the patient with computer assistance (e.g., to provide pre-modeled or planned injection points and pathways and/or to monitor distribution achieved during the transfer);

FIG. 2 is block diagram of a tissue transfer system of one embodiment of the invention illustrating functionally tools including computer software and memory (e.g., programs, algorithms, and modeled data stored and/or run from memory) that is utilized to support tissue transfer;

FIG. 3 is a flow diagram illustrating one exemplary method or process for preparing adipose tissue for transplanting into a patient for soft tissue augmentation or reconstruction;

FIG. 4 is a flow diagram illustrating an exemplary tissue transfer method useful for modeling an area of a patient's body for augmentation or reconstruction, for defining injection pathways, and for providing computer assistance in performing tissue injection/implantation and monitoring injection and distribution of tissue;

FIG. 5 illustrates a computer modeling process in simplified form showing the modeling of a virtual breast, in this example, that is to be reconstructed (or augmented);

FIG. 6 shows an injection pathway model as may be displayed on a computer monitor or provided in a print out with the model illustrating defined injection points and one or more pathways defined for each injection point to achieve a desired distribution of tissue (e.g., more uniform distribution); and

FIG. 7 illustrates a side view of the model of FIG. 6 illustrating that the pathways may have differing or equal lengths and differing angular projections relative to a horizontal plane passing through the injection guide or other a reference plane.

### DETAILED DESCRIPTION OF THE PREFERRED EMBODIMENTS

Briefly, the present invention is directed to a method and associated tools and/or systems for performing reconstruction or augmentation of a patient's soft tissue such as tissue found in a breast, face, buttocks, and the like. The method may be considered adipose-derived adult stem cell (ADAS) therapy because the method involves the transfer of donor adipose tissue, e.g., from the same patient or autologous tissue, after the tissue had been processed or purified to compose or at least be rich in ADAS. In some preferred embodiments, the purified adipose tissue is harvested from a donor site on the patient and then purified by centrifugation to separate water from the harvested tissue and oil from damaged mature adipocytes. The separated water and oil are removed from the processed tissue, and the purified adipose tissue is transferred such as through lipoinjection to an area of the patient that is being reconstructed or augmented. The method of the invention is well-suited for use in augmenting and reconstructing breast tissue, and the following discussion emphasizes this application to ease explanation of features of the invention while those skilled in the medical arts will readily understand the usefulness of the method to other bodily areas composed of soft tissue such as adipose tissue.

The tissue transfer generally involves first modeling an area to be treated such as a woman's breast. The modeled treatment surface or area is then used to define the location of a plurality of injection points to be used in injecting the purified tissue and also one or more pathways (e.g., angular trajectory from a reference plane(s) and each pathway's length or depth of insertion of the needle or cannula). The definition of the injection points and the pathways is performed by an iterative optimization algorithm in some cases. The pre-surgery, planned deposition is then displayed or provided to the physician for use in more uniformly distributing or implanting the purified tissue. The tissue transfer may be monitored such as by X-Ray, MRI, or infrared (IR) devices that monitor the injection needle or cannula or markers thereon to provide ongoing or real time feedback to the physician and/or to monitor actual distribution pathways and points for determination of the achieved distribution of the transferred tissue in the patient (e.g., to determine if additional transfer is necessary or the like).

The following discussion begins with a background discussion regarding the need for the special reconstruction techniques due to radiotherapy treatment of breast cancer, and this discussion is followed by implementations of the invention that are particularly well suited for use in reconstructing breast tissue in cancer patients. However, it is again stressed that the concepts of the invention, such as the treatment of harvested adipose tissue prior to transfer or implantation, are useful in reconstructive or augmentative surgery as well as for use in breast reconstruction or augmentation.

Presently, when a woman is diagnosed with breast cancer, the treatment may involve a lumpectomy or radical mastectomy to remove breast tissue and such tissue removal is most typically followed by radiotherapy. Radiotherapy causes permanent damage to tissues remaining in the breast, and this damage may actually worsen over time. The damage may include radiation-induced fibrosis in the subcutaneous adipose tissue of the breast ranging from mild to severe and may also include inflammatory areas or lipophagic granulomes. Capillary vessels may also be reduced in number and may have focal duplication of the basal membrane. Studies have further shown damage to adipocytes including lisosomes in the perinuclear cytoplasm or enlargement of the perilipidic cytoplasmic sheets. The radiation-induced fibrosis and other damage are limiting in the dose of radiation or otherwise dictate how radiotherapy can be provided to patients to control or reduce such damage. Previous reconstruction efforts have involved the use of implant and transfer of untreated adipose tissues, but neither reconstruction method has been fully successful. Implant recipients offer suffer ulcers that may be painful or even result in the implant being exposed. Transplant recipients of adipose tissue composed of mature adipocytes have not been effective as the tissue generally is not sufficiently vascularized which leads to necrosis and other problems and also includes a significant amount of triglycerides that may later generate granulomas. Additionally, the distribution of injected tissue is often localized or random which may lead to an irregular texture or shape and localized spots of damaged and healthy tissue. As will become clear from the following discussion, the tissue transfer method of the invention typically involves a method of purifying harvested adipose tissue of triglycerides and processing that increases the clearance or removal of damaged mature adipocytes after injection into the patient. The tissue transfer method also includes generating a computerized model for use during tissue transplant to achieve more uniform distribution by defining injection point locations and injection pathways from each of these points.

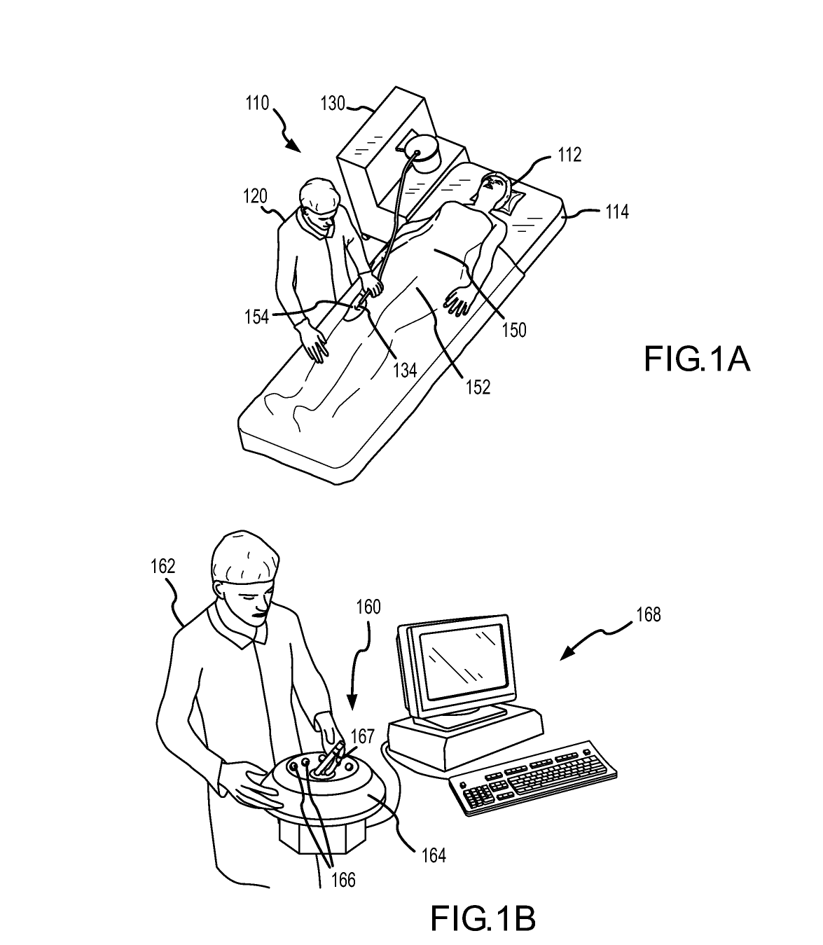

In a simplistic form, FIGS. 1A to 1C illustrate implementation of the present tissue transfer method. Referring to FIG. 1A, the tissue transfer method includes harvesting adipose tissue 110 from a donor 112 shown prone on a bed or support 114. In some preferred embodiments, the tissue transfer is an autologous tissue transfer and in these embodiments the donor 112 is the same patient that is undergoing a breast reconstruction or augmentation (as shown in FIG. 1C). A physician or technician 120 utilizes a liposuction machine 130 to extract a volume of adipose tissue via a needle or cannula 134. A variety of donor sites may be chosen for obtaining the adipose tissue such as, but not limited to, the abdomen 150, a thigh or the trochanteric area 152, or a knee 154.

Referring to FIG. 1B, the inventive method includes processing or purification harvested adipose tissue 160 to address prior tissue problems with large tissue transfer due to necrosis and other issues with transplanting adipose tissue with large amounts of triglycerides and mature adipocytes. During purification 160, a technician or operator 162 places vial or other tissue containers 166 containing the volume of harvested adipose tissue 167 in a centrifuge (or other separation device) 164. As shown, the centrifuge 164 is operated or rotated for a period of time to separate water from the tissue and a large part of the triglycerides stored in the tissue. Both of these separated components are then removed from the vials 166 (or, alternatively, the adipose tissue that has been separated from the water and triglycerides is removed for later injection into the patient 112). Interestingly, the purification method 160 does not attempt to maintain mature adipocytes in a relatively undamaged condition, but it instead is designed to further damage a significant amount of these mature adipocytes in the adipose tissue 167 to hasten their clearance by the patient's body after implantation.

For example, the centrifugation (e.g., at rates in the range of about 1500 rpm to about 4000 rpm over a time period ranging from several minutes to about 20 or more minutes) may cause lesions in the thin cytoplasmic sheets of the mature adipocytes, which favors their rapid clearance after implant. Oil or other byproducts from these damaged adipocytes is also separated from the adipose tissue and is removed to leave a purified volume of adipose tissue (or the tissue is removed from the separated water, oil, and other byproducts). The operation of the centrifuge 164 and separation/isolation of the purified adipose tissue may be controlled automatically by a controller (e.g., a computer with a monitor as shown or other electronic device with a processor and memory) 168 that may store and later run one or more purification programs or protocols previously determined to provide a desired purification result.

The tissue transfer method also includes tissue transfer or deposition 170 into the patient 112. The purified tissue from the processing 160 is believed to be better suited for successful implantation and acceptance for use in reconstruction and augmentation and, hence, all existing techniques for injecting and distribution of adipose and other cells/tissue may be used to implant the purified adipose tissue. In other embodiments, though, it is desirable to perform the transfer 170 such that improved distribution is achieved. As will become clear from the following discussion, it is believed that more uniform distribution of the adipose tissue can be achieved through the use of computer-assisted modeling of the patient's breast (or other implantation site or area) and optimization of the location of injection points and injection pathways from such points (e.g., path length and angular trajectory from the point). Further, the positioning of the injection needle or cannula or the like can be monitored during the transfer 170 to provide real time feedback to the physician 172 and/or to determine whether the modeled injection points and pathways were utilized or followed during the transfer 170 (e.g., to determine the actual points and pathways to determine the achieved tissue distribution in the patient 112).

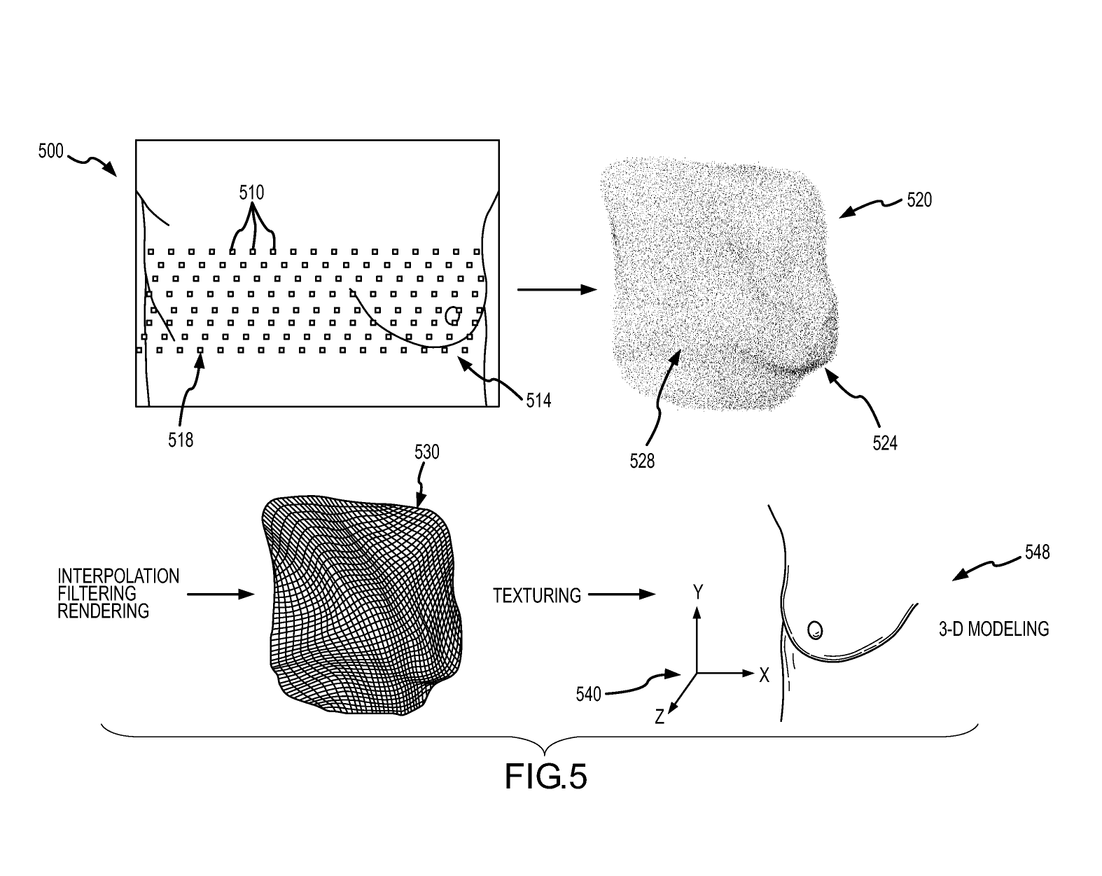

With this brief background in mind, the process 170 is shown to involve a physician 172 using a syringe filled with a volume of the purified adipose tissue 174 from process 160 to transfer the tissue 174 via needle or cannula 175 into a breast 113 of the patient 112. An injection guide 176 is positioned about the perimeter of the breast (or reconstruction/augmentation site or area) 113 to assist the physician 172 in identifying the injection points (see, also, FIG. 6 for more explanation of the use of an injection guide). The injection guide 176 may also be configured to provide the physician 172 with a reference (such as be in planar form marked with angular offset similar to a protractor) for injecting the tissue 174 along various injection pathways from each injection point. Alternatively, the injection points may be mapped and marked prior to the surgery or tissue transfer 170 to allow the physicians to locate the predetermined injection points. A control system 180 such as a computer with a monitor is provided to display the injection pathway model to the physician 172, and this may involve a three dimensional (3D) model of the breast 113 being displayed on the monitor along with all the injection points and their pathways or a “next” injection point and its pathways or a “next” pathway to be used by the physician 172 to sequentially perform the tissue deposition. A monitoring system 190 is provided with monitors/sensors 192 to track the progress of the process 170 such as by using X-Ray, MRI, IR, or other techniques along with optional markers on the guide 176 and/or needle 175 to allow the injection points and pathways actually followed by the physician 172 to be identified. In some embodiments, the injection tracking or monitoring includes provided real time imaging and display on the monitor of system 180 (or another monitor) and/or storing the information, which is later used to determine or generate a tissue deposition map that may show actual injection points and pathways and/or variance from the injection point and pathway model created prior to the procedure 170.

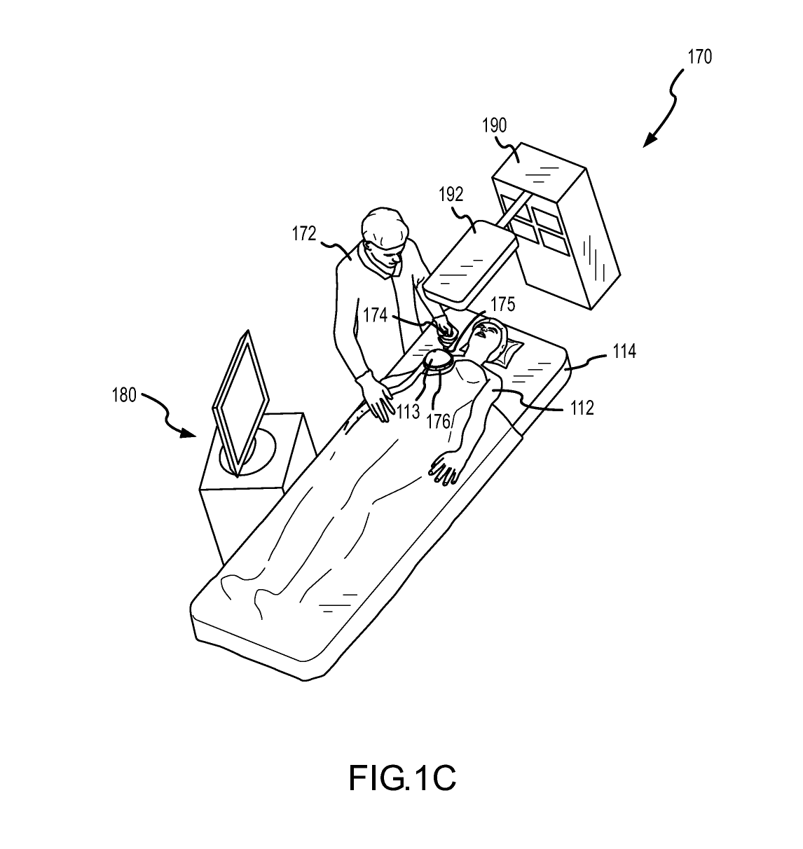

An embodiment of the tissue transfer of the present invention may be thought of as an autologous transplant of lipoaspirate (e.g., tissue such as adipose tissue harvested or aspirated via liposuction or similar methods from a patient). As noted, one of the significant uses of this inventive transplant method is for tissue regeneration in an area of a patient's body that is suffering from radiation-induced lesions following radiation therapy. To this end, FIG. 2 illustrates in functional block form that in addition to harvesting or obtaining a volume of adipose tissue that the transplant system or tools 200 includes a surface modeling system 210, a tissue purification system 240, and a tissue transfer system or site 260 with each including its own tools, devices, and systems for achieving a desired end function.

Rather than using random injection or best judgment attempts to avoid clump injections, the surface modeling system 210 is used to generate a map or model of optimized injection points and pathways. To this end, the system 210 includes a processor or CPU 212 that receives and processes input from one or more cameras 214 and, optionally, a manual scanner or sensor 216. The camera 214 and sensor 216 may be used to obtain digital images 222 of the site to receive the implant such as breast that has undergone treatment for cancer and to obtain images 222 of a healthy breast such as the patient's breast that has not been treated for cancer. The images 222 are stored in memory 220 or otherwise made available to the processor 212. Alternatively, the images 222 may be obtained from other women or patients to obtain a digital image 222 of an area or surface to be reconstructed or augmented, e.g., to obtain images of a breast for which augmentation is being used to obtain or when a patient has had damage to both breasts and cannot provide images 222 for use in reconstruction. The processor 212 runs a modeling algorithm 218 to process the images 222 to generate a breast model (or model of another bodily area or surface to be reconstructed or augmented) such as by generating a mirror image of the patient's normal or undamaged breast or via manipulation of an existing breast as is known by those skilled in the reconstructive and cosmetic surgery fields for modeling bodily features after reconstruction or augmentation.

With the breast model 224 in memory 220, the processor 212 next runs an injection optimizer 230 that uses one or more optimization algorithms to process the breast model 224 and a set of optimization variables 226 to determine a set of injection point locations and pathways from such injection points 228 (as may be defined by length and angular trajectory from the injection point). The variables or parameters 226 may include a maximum number injection points to be utilized and a number of pathways from each point. Typically, the volume of tissue to be injected via each pathway is predetermine or fixed (e.g., such as the overall volume of purified tissue to be deposited or transferred divided by the number of injection pathways although, of course, the volume of tissue may be varied at each injection point and/or along each pathway to practice the invention and achieve a desired distribution in a breast).

The surface modeling system 200 provides computer-assisted, patient-specific planning of lipoaspirate surgical deposition. The planning or assistance is achieved with the mapped or determined injection point locations and defined pathways 228, which, as discussed above, are generated based on a computerized 2D or, more typically, 3D model of the body area to be treated. The model 224 is obtained by digital and calibrated photographs or images and/or laser scanning images from the cameras 214, scanner 216, or other equipment (not shown). Computer-assisted, pre-surgical planning of lipoaspirate deposition is designed to achieve near maximum uniformity of distribution and to limit significant overlaps and gaps in the tissue deposition. The process performed by the optimizer 230 is based on constrained optimization methods with the constraints or variables 226 in some cases including numbers and initial position of entry points, feasible length of insertion pathways (as a function, for example, of the length of the cannula(s) planned for use in the deposition and often based on the size of the bodily area being augmented or reconstructed), peak angular values of feasible insertion pathways, and, in some cases, inaccessible or untreatable areas. In some preferred embodiments, these variables 226 are patient specific and are selected by a physician or other technician to suit the particular patient and their needs and/or body shape and configuration (e.g., are entered into memory 220 as a step of the modeling process prior to optimization by the injection optimizer or algorithm 230).

Patient-optimized surgical planning carried out by the system 210 via running optimizer 230 may include minimizing or reducing a constrained objective function that is designed to minimize or limit dimension and variability of the areas generated by the intersection of tissue deposition pathways (see, for example, FIG. 6). The mapped injection model 228 produced is or may be used to generate a composite representation of the optimized entry point positions and directions of the insertion pathways from these points superimposed upon select patient images 222 and/or 3D digitized model 224 (again, see, for example FIG. 6). An advantage of the modeling system 210 and its implemented processes is that they make available an interactive optimization process for tissue deposition. For example, a technician or physician may interact with the system 210 during the creation of the model 224 to obtain a desired result after reconstruction or augmentation and also during selection or setting of the optimization variables 226 (e.g., changing the number of injection points, the number of pathways, or the like). Such pre-surgical planning can lead to standardization of the surgical procedure rather than relying on the judgment, experience, and skill level of the surgeon and can produce pre-surgical quantitative parameters. This ultimately reduces uncertainties in clinical outcomes between differing patients and produces patient-related quantitative documentation on the achievable accuracy in tissue deposition.

The system or set of tissue transfer tools 200 includes a tissue purification system 240 for processing or purifying adipose tissue prior to use for an implant. As shown, harvested adipose tissue 242 is provided to or positioned in a separation device such as a centrifuge 244. The centrifuge 244 may be manually operated or run automatically by a controller 246 based on a purification protocol 248 (e.g., a program defining one or more centrifugation speeds and times). During this processing, a portion of the tissue such as water, oil from damaged mature adipocytes, triglycerides, and other components, separate from other adipose tissue. This volume or portion is removed 256 leaving a volume of purified adipose tissue 250. For example, the protocol 248 may define a harvested volume to be inserted into each reservoir or vial in a centrifuge 244, a revolution rate at which to run the centrifuge, and a time period. After the time period ends, the separated, undesired portion 256 is removed from each reservoir or vial in the centrifuge 244. Alternatively, the protocol 248 may define a stepwise procedure and define volumes of the separated tissue 256 to be removed at each step (e.g., run the centrifuge at a first speed, remove a particular volume of separated tissue 256, run the centrifuge at a second speed, remove another volume of tissue 256, and so on) or such removal may be handled automatically with some centrifuges 244 or separation devices being configured to selectively remove the separated tissue 256 or to remove the purified adipose tissue from a “ring” in the centrifuge reservoir or vials (e.g., based on expected location of the purified adipose tissue 250 based on centrifugal rates and densities of the tissue 250).

Significantly, in addition to harvesting the adipose tissue, the procedure for procurement and treatment of autologous (or other donor) adipose tissue or lipoaspirate includes purifying the tissue. The lipoaspirate purification procedure is generally designed to remove a large part of the triglyceride stored in the harvested adipose tissue. The purification by centrifugation or similar techniques also functions to cause lesions in the thin cytoplasmic sheets of mature adipocytes in the harvested adipose tissue. In other words, the purification includes intentionally causing additional damage to the adipocytes that have been traumatized by liposuction or harvesting processes, and this additional damage is preferably to the point of one or more lesions so as to enhance the speed at which a treated patient is able to clear the damaged mature adipocytes after implant. In some preferred embodiments, purification is obtained by centrifugation carried out, in part, to separate a set of adipose tissue (i.e., the purified adipose tissue) from its water content and from the oil produced by the destruction of the damaged adipocytes. An advantage of use of the inventive purification technique is that there is no need for any kind of cell culture to grow additional tissue outside the patient's body as was common with many other tissue implant techniques, and avoiding culturing better controls risks of micro-organism contamination, reduces the complexity of the tissue preparation process, and controls or limits associated costs. A further advantage of the purification or tissue preparation process is that by the process does not require the technically challenging step of isolating or extracting adipose-derived stem cells (ADAS) but instead allows the ADAS to remain in their natural support structure or 3D scaffold which facilitates vascularization and other benefits.

The system 200 also includes a tissue transfer site or system 260. The system 260 includes a processor or CPU 280 that acts to provide computer assistance to a physician during or before tissue transfer. The processor 280 functions to access memory 290 and to display a distribution model such as by displaying on the monitor 288 (or providing a hard copy) the modeled injection point locations and pathways 292 or superimposing this information 292 upon a 3D or 2D model of a breast or other portion of a patient's body. As shown, the tools or system 260 includes an injection guide 270 with indicators 274 showing modeled injection points to provide a reference point and plane for performing the injections. The injections or tissue transfer is performed in this case with a syringe 262 or similar device that contains a volume of purified adipose tissue 264 from purification system 240. Using the modeled injection distribution on the monitor and the guide 270 a physician (not shown) performs the tissue deposition by inserting the needle or cannula 266 at each injection point (e.g., points marked or referenced on the patient or by indicators 274 of guide 270) and attempting to follow defined pathways. An injection pathway monitor 284 is provided to determine such as by identifying the location of a marker(s) 268 on the needle 266, the pathways actually used by a physician during tissue deposition or transfer. This information from the injection pathway monitor 284 may be stored with or without further processing by processor 280 as shown by achieved distribution data 294 in memory 290.

With the tissue transfer site or set of tools 260, the system 200 provides computer-assisted, intra-surgical guidance for lipoaspirate deposition. The pre-surgical plan including the injection points and pathways 292 provides the surgeon with the map for intra-operative guidance or aiming to achieve a high level of uniformity of adipose tissue deposition, which is typically purified adipose tissue but the pre-surgical plan would benefit nearly any tissue transfer or implant such as unpurified adipose tissue, adipose tissue with additional stem cells, or other tissues/cells (e.g., mesenchymal cells, especially smooth or skeletal muscle cells, myocytes (muscle stem cells), chondrocytes, adipocytes, fibromyoblasts, ectodermal cells, or nerve cells which may or may not be dissociated). Further, growth factors, angiofactors, anti-inflammatories, selective growth inhibiters, and the like may also be provided with or after implantation of the tissue. Tissue and cells are preferably autologous cells, obtained by biopsy and expanded in culture, although cells from close relatives or other donors may be used such as with appropriate immunosuppression. Immunologically inert cells, such as embryonic cells, stem cells, and cells genetically engineered to avoid the need for immunosuppression may also be used. Yet further, tissue expanders may be useful in some applications but are generally not a required tool for use with the present tissue transfer method.

A specific interactive tool such as the system 260 allows an operator to select (such as via a mouse, a keyboard, a touch screen, by voice command or other user interface or user input device) a specific entry or injection point and pathway at that point and to proceed pathway-by-pathway and point-by-point to complete the network of predefined pathways (such as via a display of model 292 via processor 290 on monitor 288). Additional features of the tool set 260 include the monitor 284 that may be used by the processor to provide a surgical navigation system for guiding the surgeon during cannula 266 insertion. Such a monitoring system 284 may be based on an optical IR real-time tracking device, which provides the 3D position of the cannula 266 (e.g., carrying a configuration of IR reflecting markers 268 or the like) with respect to the stereotactic patient-mounted reference frame or model shown on the monitor 288 upon which the surgical plan 292 may also be superimposed or mapped. In this embodiment, a real-time graphic feedback is generated on the displayed surgical plan (e.g., tissue transfer distribution mapping) at the computer screen 288 providing information on the current deposition direction and, in some cases, signaling deviation with respect to the planned trajectory or pathway and, in some further embodiments, providing a related correction. The intra-operative method ensures improved accuracy in transferring the planned injection points and pathways 292 into the reality of the surgical procedure and also produces specific quantitative documentation 294 describing the actual geometry of lipoaspirate that the operator or surgeon was able to achieve for the particular patient.

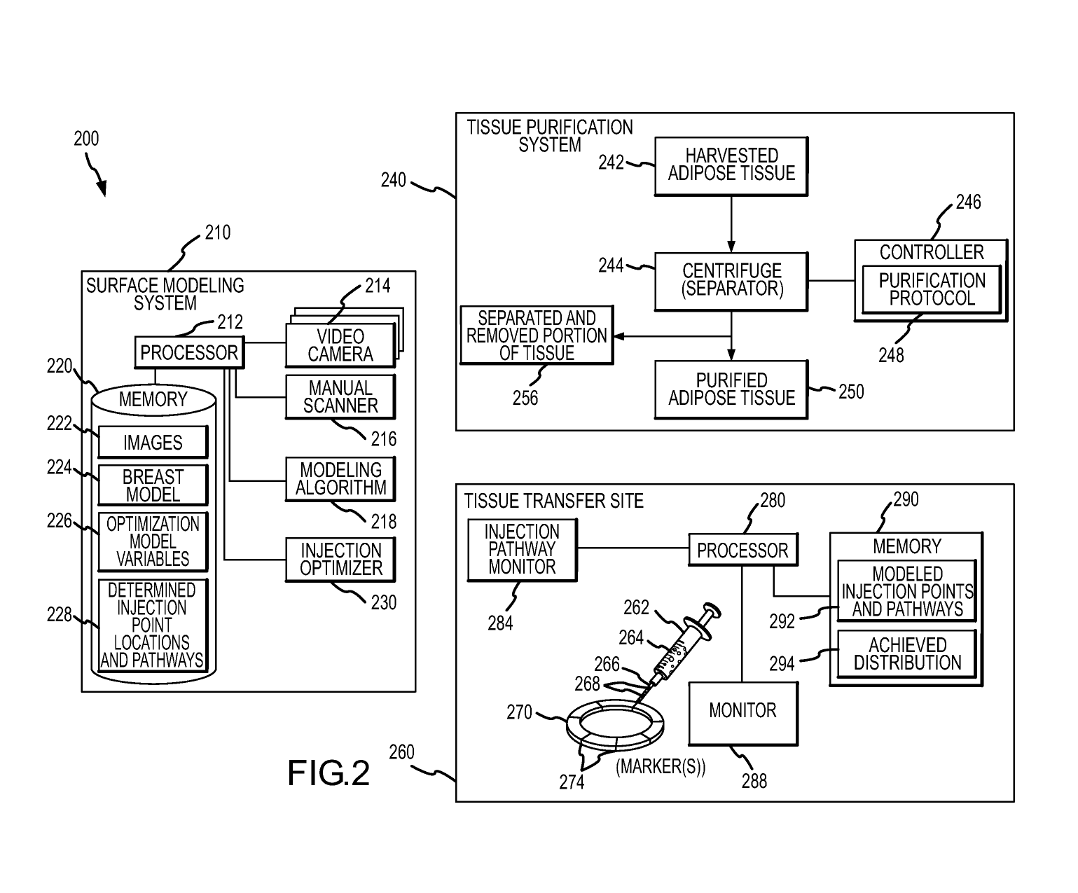

FIG. 3 illustrates generally the steps of a tissue preparation process 300 that starts at 305 and is used to prepare a volume of adipose tissue for implanting or deposition in a patient such as for augmentation or for reconstruction of soft tissue after removal and/or radiotherapy. The method 300 continues at 310 with the selection of a donor and donor site. As noted above, the tissue is typically autologous tissue but this is not a requirement of the invention. In step 310, a donor site for obtaining adipose tissue is selected such as the medial area of the knee, the abdominal region, the trochanteric, or other regions of the donor's body. At 320, the donor site is prepared for harvesting such as by infiltrating the selected region with a cold saline solution with the addition of adrenaline (e.g., 10 to 20 cubic centimeters (cc)) and lidocaine (e.g., 20 to 30 cc of lidocaine 0.5% per 500 cc or the like). At 330, a volume of adipose tissue (e.g., up to 2 or 3 cc of adipose tissue or more) is removed such as by using a cannula (e.g., a 2 mm or other diameter cannula) and a syringe.

At 340, the harvested adipose tissue is transferred to a centrifuge for centrifugal separation or purification such as by placing a plurality of syringes directly in the centrifuge or transferring their contents into different reservoirs or vials. In some embodiments, the operator is allowed to select a purification protocol from a set of previously determined useful protocols while in other cases a default or preferred protocol is set or fixed for use in all purification steps 350. In some cases, for example, the protocols may include (but are not limited to): (a) a spin speed or centrifuge rate of about 1900 rpm for a spin time of about 15 minutes; (b) a spin speed of about 2700 rpm for about 8 minutes; (c) a spin speed of about 2700 rpm for about 15 minutes; (d) a spin speed of about 3500 rpm for about 8 minutes; and (e) a spin speed of about 3500 rpm for about 15 minutes. More generally, the protocol may be thought of as operating the centrifuge at a spin speed and for a spin time predetermined to obtain substantial separation of water from the tissue, oil from the damaged mature adipocytes, triglycerides, and/or other undesired components and the spin speed typically is in the range of about 1000 rpm to about 4000 rpm or higher but more typically between about 1900 rpm and about 3500 rpm and the spin time ranges from several minutes to about 30 or more minutes but more typically is in the range of about 8 minutes to about 15 minutes. The preferred protocol is generally one in which achieves substantial removal separation of the oil upon causing lesions in a significant percentage of the mature adipocytes and separation of substantial triglycerides while retaining structural integrity of stem cells (e.g., maintains cell viability of ADAS to a large degree). At 360, the centrifuge loaded with the harvested adipose tissue is operated based on the selected or default protocol. At 370, the separated oil, water, triglycerides, and/or other components or tissue separated from the adipose tissue is removed to generate a smaller volume of purified adipose tissue (e.g., tissue composing or being rich in ADAS). The purified adipose tissue is, at least temporarily, stored or packaged in step 380, for later transfer to a patient (e.g., the donor), and the process 300 ends at 395. The overall volume of purified adipose tissue may vary widely to practice the invention and typically with each patient. As an example, the average size of a breast implant is in the range of about 325 to about 400 cc, and it may be desirable to prepare up to about 400 cc or more of purified adipose tissue to perform a breast reconstruction after a full mastectomy followed by radiography treatment.

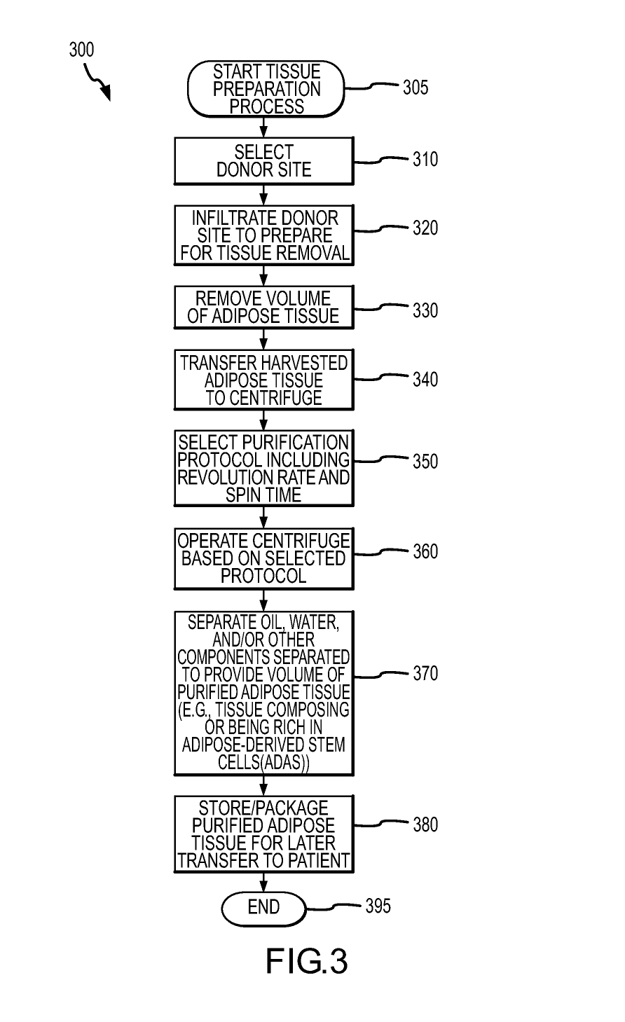

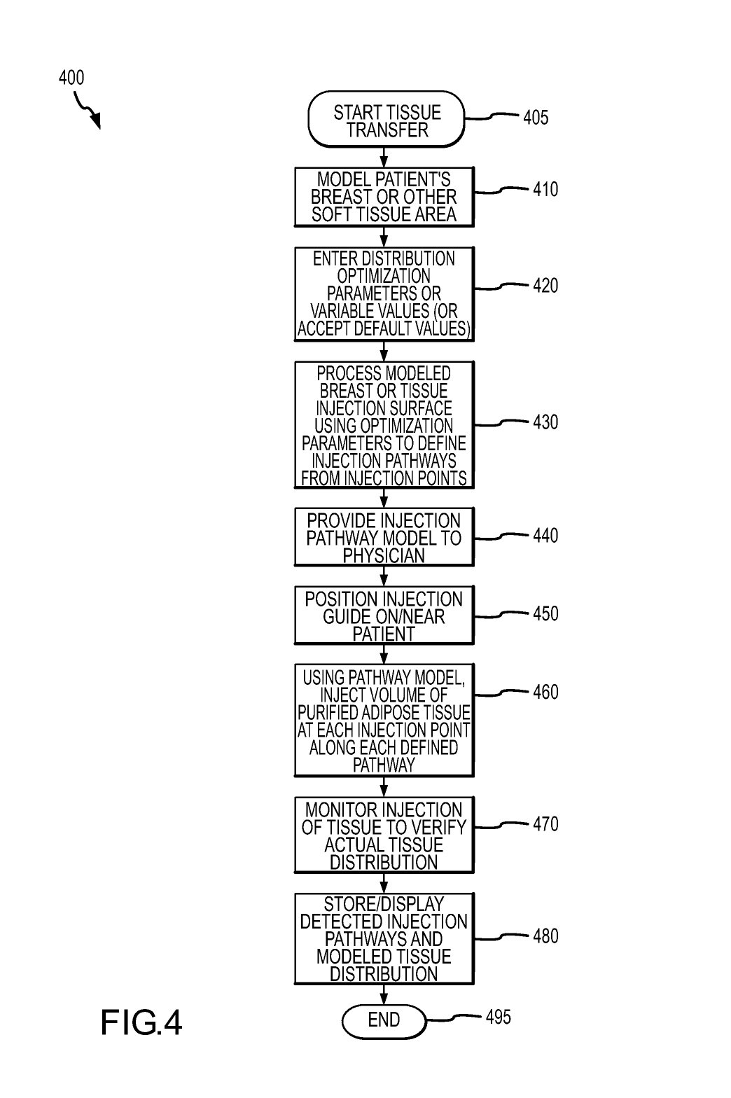

FIG. 4 illustrates exemplary steps of a tissue transfer or deposition process 400 of the present invention that starts at 405. At step 410, the area of the patient (e.g., one or both of the patient's breasts) that is being augmented or reconstructed after loss or damage of soft tissue such as adipose tissue it modeled. Such modeling 500 is shown relatively generally in FIG. 5 and includes obtaining one or more photographs of the area such as of the area to be reconstructed or augmented 518 and a reference area 514 (e.g., the patient's other breast). In some cases, a manual or laser scanner may be used instead of the photographs or in addition to the photographs to obtain a plurality of data points 510 indicative of the 3D topography of the reference breast or area 514 and the area to receive the implant 518. The image is digitized as shown at 520 to provide a digital image or plurality of data points of the reference area 524 and of the implant area or site 528. Interpolation, filtering, and rendering are used to generate a more complete computer model of the reference breast or area and of the area to be augmented or reconstructed as shown at 530. Then, texturing and other processing is performed to achieve a 3D model or virtual version 540 of the breast to be reconstructed or augmented 548, which is typically the desired or final form for the breast or body area and may be a mirror image of the reference breast or area or may be a modeled or textured model or plan for the breast or area being reconstructed or augmented.

At 420, the method 400 continues with entering distribution optimization parameters or variable values or alternatively accepting one or more default values. The parameters or variables typically include at least a number of injection points and a number of injection pathways at each injection or entry point. The parameters may also include a maximum length of the pathways and can sometimes include a maximum or peak angle for the pathway. At 420, the method 400 continues with processing the modeled breast or tissue injection surface from step 410 using the optimization parameters of step 420 to define preferred or “optimized” injection pathways from a set of injection points, with the injection point locations also being defined. The model or planned injection mapping/network is stored in memory, and at 440 is provided to a physician for use in performing tissue transfer or deposition. The model is typically overlayed or superimposed on the modeled breast from step 410, and the model is often provided on a computer or other monitor in the operating room. At step 450, an optional injection guide is positioned on or near the patient such as about the perimeter of the breast or other area to be reconstructed or augmented. The guide is optional as in some cases it is preferable to mark or otherwise identify the injection points from the model on the implant site. At 460, the pathway model is used to inject a volume of purified adipose or other tissue at each injection site and along each defined pathway. Step 470 is optional and provides for monitoring of the tissue transfer or injection of step 460 to provide injection guidance and/or verification/documentation of actual tissue distribution. The monitored or detected injection pathways and the modeled tissue distribution (calculated actual distribution) may then be stored in computer memory at 480, and the process 400 ends at step 495.

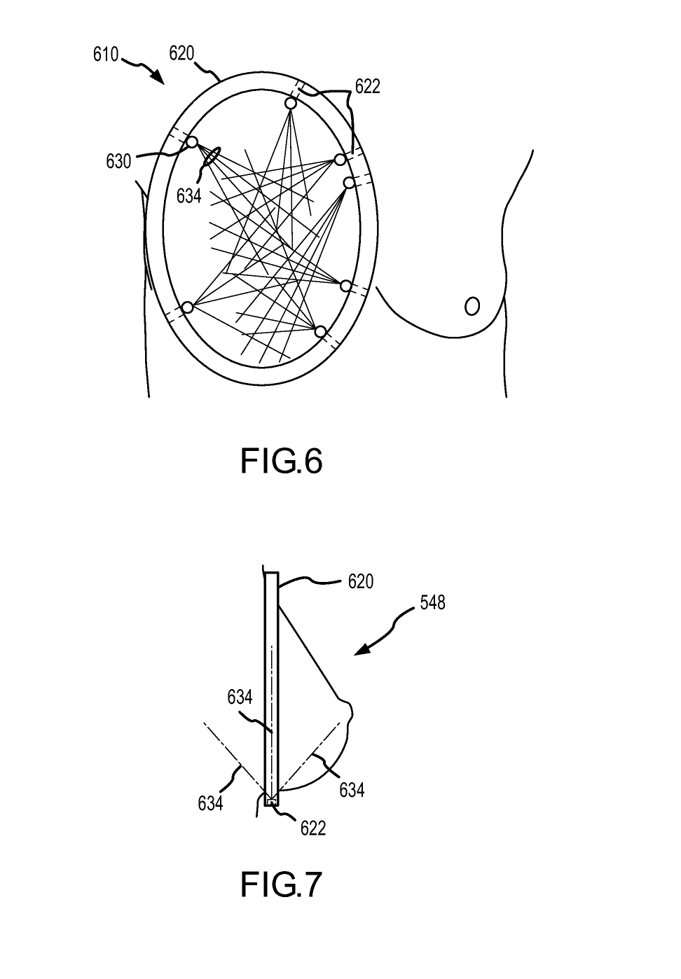

FIGS. 6 and 7 provide an illustration of an exemplary injection pathway model 610 as may be displayed on a monitor or otherwise presented to an operator or surgeon and is shown when in use with an injection guide 620 with injection point indicators or reference lines 622. A plurality of injection points 630 are spaced apart about the periphery of the implant area (e.g., a patient's breast). As shown, the points 630 are not equally spaced but instead have been located in a more irregular pattern by the optimization algorithm to achieve better distribution. Also, the periphery or outline defined by the points 630 is shown to be relatively circular, oval, elliptical or the like but often the periphery will be an irregular shape. As shown, the optimization parameters include a number of points of seven and the number of injection points at each point was set at four. Of course, smaller or larger values may be used for each of these parameters or variables. Also illustrated in FIGS. 6 and 7 is the feature that the travel pathways 634 do not necessarily have equal lengths and injection by the surgeon at each pathway may require a reference marking on the needle/cannula or differing length needles/cannulas to match these lengths. Further, each of the injection pathways 634 is spaced apart and is defined by a corresponding trajectory angle (positive or negative) from the injection or entry point 630 which may be relative to a plane passing through the injection point horizontally and vertically (e.g., a 3D trajectory path is defined for each pathway), and, in some cases, the guide 620 provides the horizontal reference plane for the trajectory pathways 634.

In one embodiment, the computer optimization algorithm performs multi-parametric optimization through non-linear, unconstrained minimization (e.g., in a projective 2D version). Input in this case may be the surface model of the implant area or site, the number of entry points (e.g., 3 to 10 or more), the number of paths per entry point (e.g., 1 to 5 or more), and pathway length (e.g., a fixed length for every pathway in some embodiments). The output of the program or algorithm is the entry point position on the surface model (e.g., on the patient's implant site) and the path directions for all the injection points. The function cost is typically made of: the number of areas in which the paths overlap (to be maximized in most cases), the area dimensions (to be minimized in most cases), and the area variability (to be minimized in most cases). In practice, the algorithm generally works by starting from an initial guess and then iteratively searching for the “best” position with respect to the model of the implant site surface for: entry points, path direction, and, in some cases, path length. In some preferred cases, the “best” position is selected in order to maximize the number of areas formed by the intersections of the injection pathways and to minimize their absolute size and size variability (i.e., homogeneity).

As described, the tissue transfer methods and tools of the invention provide enhanced techniques for preparing adipose tissue for implanting in a patient and also an enhanced computer-assisted model for performing tissue deposition. The above outlined tissue preparation and transfer may be used in many tissue augmentation and reconstruction situations and is not limited to a particular treatment or surgical procedure. However, the inventor has noted that on the bases of pathogenic considerations generated by the detection of scleroderma-like chronic microangiopathy, the methods and tools described herein are particularly suited for autologous transplant to treat women who have received radiotherapy and is based on injection of tissue enriched with adipose-derived adult stem cells (ADAS) or purified adipose tissue. With this in mind, a clinical trial was performed using the innovative therapeutic approach aimed at minimizing or controlling the radiotherapy-related morbidity based on injection of autologous ADAS.

In the clinical trial, 20 patients who underwent adjuvant radiotherapy for cancer and who presented a radiolesion classified on the LENT-SOMA scale as Grade 3 or severe symptoms or Grade 4 or irreversible function damage but had no medical history of connective, metabolic, or skin disease. The areas that were damaged by radiotherapy and were treated using purified adipose tissue or ADAS-rich tissue (as described above) included the supraclavicular region, the anterior chest wall (i.e., the site of a masectomy). Fourteen of the twenty patients had breast prothesis inserted as part of the initial or pre-trial reconstruction of the breast. In the group of eleven patients with Grade 4 radiodamage, the lesion involved the chest wall in 8 cases, the breast in 2 cases, and the supraclavicular area in 1 case. Fibrosis, atrophy, and retraction were classified as Grade 4. Besides the 8 patients with chest wall lesions, 4 patients had silicon gel breast implants. Three of these 4 patients presented ulcers causing exposure of the implant and 1 patient had telangectasia involving an area of skin greater than 4 square centimeters. Of the other 4 patients without implants, 1 patient had an ulcer exposing osteoradionecrotic ribs and refractory pain and 1 patient had telangectasia involving an area greater than 4 square centimeters. Of the 2 patients with breast lesions, 1 patient presented an ulcer and the other telangectasia. In the group of 9 patients with Grade 3 radiodamage, the lesion involved the breast in one case and the chest wall in the other eight cases. Four of these patients had silicon gel breast implants. All of these patients presented Grade 3 atrophy, fibrosis, and retraction. In addition to these symptoms telangectasia and pain were experienced by some of the patients.

The tissue transfer process included selecting an area as the donor site (e.g., the medial area of the knee, the abdominal area, or the trochanteric area) and then infiltrating the area with a cold saline solution with the addition of about 15 cc of adrenalin and about 20 to about 30 cc of lidocaine 0.5% per 500 cc. Adipose tissue was removed using a cannula with a 2 mm diameter and a 3 cc syringe. The syringes were placed directly in a centrifuge that was then set at about 2700 rpm and run for 15 minutes, which resulted in separation of the purified adipose tissue for injection from its water content and from oil resulting from the destruction of damaged adipocytes. The later of oil and residual liquid (including triglycerides) was then discarded. The adipose tissue was implanted in the same patient using an injection cannula with a 1 mm diameter in single tunnels or pathways made by following a pre-surgical plan or model of the injection points and pathways (as discussed in detail above) to ensure substantially uniform distribution of the ADAS or purified adipose tissue.

The computerized model for injection provided a plan for the surgical procedure of tissue transfer and deposition and was planned with a computer that ran an iterative optimization algorithm. The aim was to acquire quantitative pre-operative information concerning the optimal positioning of access points as well as the number and direction of insertion pathways. A goal was to allow the surgeon to achieve maximum or at least improved uniformity of distribution and to limit significant overlaps (which can cause lumps or inadequate vascularization) and gaps in the tissue deposition. The variables to be optimized in the optimization process were entry point positions and direction of tissue insertion pathways. Both were expressed with respect to an anatomical, patient-mounted reference frame or guide. A maximum number of entry points and pathways as well as peak angular values of feasible insertion pathways was set as the boundaries of the optimization procedure. The optimal parameters were identified iteratively by way of a multidimensional, unconstrained nonlinear minimization. The objective function of the algorithm was designed to minimize dimension and variability of the areas generated by the intersection of tissue deposition pathways associated with each set of parameters. The output of the optimization procedure included entry point positions and insertion pathway directions providing high uniformity of tissue deposition under the pre-defined set of boundary parameters. The level of algorithm convergence and the residual value of the objective function correlated with the expected geometrical quality of the surgical procedure. It will be understood that increasing the number of entry points and pathways at each point increases the degree of homogeneity of the tissue distribution but at the cost of increasing the complexity of and time required to complete the tissue transfer or deposition.

After tissue transfer based on the injection model or plan and using autologous, purified adipose tissue, in the 11 patients classified as Grade 4, four patients progressed to Grade 0, five patients progressed to Grade 1, and two patients progressed to Grade 2 with regard to fibrosis, atrophy, and retraction. With regard to the 5 patients with ulcerations, 2 of the 3 patients with breast implants, for whom the ulcerations had led to exposure of the prosthesis, experienced healing of the lesion after the transplant of ADAS and resuturing and with conservation of the implant. In the other patient, the treatment was unsuccessful with extrusion of the prosthesis. In the patient without a breast implant who had ulceration in the chest region, the purified adipose tissue transfer resulted in excellent granulation of tissue that was later covered with a skin graft. In the patient with an ulcer on the breast, the lesion healed. In the patient with telangectasia and retraction in the supraclavicular area, the telangectasia as well as the pain disappeared. In the 5 patients with telangectasia, there was total resolution (i.e., to Grade 0) in 2 cases and a significant reduction in vascular diameter and chromatic intensity (i.e., to Grade 1) in the other 3 cases. In the 9 patients classified Grade 3, the fibrosis, atrophy, and retraction progressed to Grade 0 in 5 patients and to Grade 1 in the other 4 patients. Finally, in the patient with telangectasia, complete healing was achieved with a total remission of symptoms causing pain.

These results are very promising and show, at least initially and on a small scale, the potential efficacy of the tissue transfer techniques taught within this description. As noted earlier, previous experiments performed by others had demonstrated that transplants of adipose tissue composed of mature adipocytes are not sufficiently vascularized resulting in necrosis and other problems. The inventor determined also that a great amount of triglycerides present in the transferred tissue generated unwanted granulomas. Hence, the tissue transfer method includes a purification procedure that is aimed at removing a large part of the triglycerides stored in the harvested tissue. The separation, such as by centrifuge or other devices, is believed to be beneficial, in part, because it causes a lesion in the thin cytoplasmic sheets of the mature adipocytes (or otherwise causes them further damage in addition to the harvesting procedure) favoring their rapid clearance after injection in the patient. Using this approach, it was possible to inject a tissue enriched of or with a higher percentage of stem cells relative to typical or unprocessed adipose tissue. This technique is likely preferable to a disassociation of the tissue to avoid loss of stem cells. The relatively simple purification process proposed herein also reduces the risk of contaminations with micro-organisms associated with cell culturing. In addition, the stem cells or ADAS were maintained in a natural or existing 3D scaffold or support structure that in principle appears to favor reconstruction of a microvascular bed. Ultrastructural examination of the adipose tissue performed after the purification procedure confirmed these beliefs or hypotheses as it revealed well-preserved elements in the vasculo-stromal component, which was composed of endothelial cells and mesenchymal stem cells in perivascular sites. Residual mature adipocytes remaining in the purified adipose tissue showed interruptions of the cytoplasmic membrane and presented various degrees of degeneration ranging up to cellular necrosis.

Additionally, studies have been performed on tissue treated with the ADAS or purified adipose tissue of the present invention. After 1 month, subcutaneous tissue was of normal morphology and the adipocytes generally appear well conserved. The processes that remove injected material were advanced, and it was possible to find isolated lipid droplets in the fibrous connective tissues where removal is probably the slowest. Macrophages or lymphatic cells were occasionally found. The treated tissue generally appeared better hydrated than in non-treated patients. The spaces between adipocytes were large and with little collagen. Blood vessels were highly activated and showed aspects of hyperpermeability and reduplication of the basal membranes. There were elements having the characteristics of maturing pre-adipocytes (i.e., elongated or rounded, relatively poorly differentiated cells with an abundance of polyribosomes and lipid droplets). A basal membrane provided some evidence that these pre-adipocytes belong to the adipocyte line. Capillaries were present that were likely newly formed because their basal membranes did not show reduplication and because their appearance was normal in contrast to that of the vessels found in areas treated with radiation. The overall picture was characterized by signs of the removal of the injected material along with signs of regeneration. Phenomena indicating regeneration included the maturation of stem cells into both adipocytes and vascular cells. The pre-adipocytes seemed more mature a month after treatment than the pre-adipocytes found in the tissue ready for injection (i.e., the purified adipose tissue). The pattern was suggestive of an old microcirculation recognizable from lesions due to radiotherapy co-existing in the same tissue with a newly-formed microcirculation.

After two months, the processes which remove injected material were further advanced with an almost complete absence of cell debris. Macrophages and lymphatic cells were rarely found. The tissue appeared hydrated although areas of fibrosis were occasionally found. The spaces between adipocytes were large with little collagen. The adipocytes appeared well conserved. Blood vessels showed only occasional signs of hyperpermeability or reduplication of the basal membrane. The overall picture showed an end to the removal of material. The effects of radiation were still visible in the tissue, but regenerative phenomena were at an advanced state as shown by the presence of almost mature multilocular adipocytes. The absence of reduplicated blood vessels indicated that the newly formed microcirculation was advanced. At four to six months, the processes that remove injected material had finished. Very few cells were found in the connective, which seemed well hydrated with very little collagen. The adipocytes were normal. Maturing adipocytes were no longer evident. The microvessels had a normal ultrastructure with a very low percentage of vessels showing reduplication of the basal membrane. Areas of fibrosis were found in one case, though. The overall picture seemed very good or promising with no signs of removal of material. The tissue was well hydrated and the newly formed microcirculation showed no lesions. Old vessels were found only in the rare remaining areas of fibrosis. After one year, the picture remained substantially unchanged apart from some tendency towards shrinkage of the extracellular spaces. The adipocytes were large and the overall appearance was of mature adipose tissue with a well-formed microcirculation.

Observation of large populations of irradiated patients has demonstrated that issues with microvascular patterns does not spontaneously improve and typically evolves toward fibrosis without treatment. Simplistically, radiotherapy generates a fibrous adipose tissue with areas of scarring. Therapy according to the present invention with purified adipose tissue rich in ADAS leads to profound modifications in the damaged tissue that can be evaluated both clinically and at the ultrastructural level. In the early stages after therapy with ADAS, there appears to be a “mesenchymalization” of the tissue, which appears well hydrated and with large extracellular spaces resembling fetal connective tissue. Subsequently, the tissue matures presenting aspects similar to those of normal mature adipose tissue. Administration of the vasculo-stromal component, rich in stem cells, of normal adipose tissue is, therefore, capable of improving the structure of irradiated tissue.

It has been hypothesized that in an early stage the stem cells of the purified adipose tissue target the damaged areas. In a second or later stage, the stem cells likely excrete angiogenic factors that leads to the production of new microvessels that, in turn, hydrate the tissue as newly formed vessels tend to be hyperpermeable. The chain of events leading to “mesemchymalization” of the tissue, hence, appears to be: targeting of damaged areas by stem cells (which is favored by direct and uniform injection into the damaged areas as compared with clump or less uniform distribution techniques); release of angiogenic factors; formation of new vessels; and hydration. This process seems to favor the development of ADAS in mature adipocytes. After transplantation of the purified adipose tissue, a newly formed microcirculation replaces the existing, seriously damaged microcirculation. Damaged vessels can still be found in areas of fibrosis, and this emphasizes the importance of the use of multiple injection points with 1 to about 4 or more injection pathways at each point to obtain homogenous effects throughout the radiodamaged tissue. Briefly, therapy using the tissue preparation and transfer methods described herein appears to be capable of treating radiotherapeutic lesions by acting on the devascularization from which they originate and which leads to their tendency to progress.

Although the invention has been described and illustrated with a certain degree of particularity, it is understood that the present disclosure has been made only by way of example, and that numerous changes in the combination and arrangement of parts can be resorted to by those skilled in the art without departing from the spirit and scope of the invention, as hereinafter claimed. The description provided specific examples of use of the prepared adipose tissue and the injection methods for reconstructing breast tissue treated with radiotherapy. However, adipose tissue purified according to the innovative methods may be used for reconstruction of other body areas such as the face, buttocks, and other areas where soft tissue such as adipose tissue has been removed or damaged. Further, the purified tissue may be used for augmentation procedures, such as breast augmentation or the like, that utilized tissue transfer. Similarly, the injection modeling techniques may be used for nearly any tissue transfer procedure for which uniform tissue deposition is desired and is not limited to reconstruction or augmentation of breasts.

What is claimed is:

1. A method of reconstructing a breast of a patient after radiation therapy, comprising:
generating a model of a plurality of injection points and injection pathways at each of the injection points for the breast, wherein the model defines locations of each of the injection points and a direction for each of the injection pathways;
purifying a volume of adipose tissue by removing a volume of triglycerides from the adipose tissue and a volume of oil from damaged, mature adipocytes in the adipose tissue; and
transferring the volume of the purified adipose tissue into the breast of the patient by implanting a portion of the volume of the purified adipose tissue based on the defined injection points and injection pathways of the generated model.

2. The method of claim 1, wherein the model generating comprises generating a 3D surface model of the breast based on a digital model of the patient's other breast and overlaying the injection points and injection pathways on the 3D surface model.

3. The method of claim 1, further comprising before the transferring, positioning an injection guide about the periphery of the breast that is configured to provide a physical reference for each of the locations for the injection points.

4. The method of claim 1, further comprising harvesting the volume of adipose tissue from the patient and wherein the purifying comprises centrifugation at a spin rate greater than about 1000 RPM to cause damage to at least a portion of the mature adipocytes in addition to damage caused by the harvesting from the patient.

5. The method of claim 4, wherein the damage caused by the centrifugation comprises a lesion in a cytoplasm sheet of the portion of the mature adipocytes.
